(19) United States
(12) Patent Application Publication
(10) Pub. No.: US 2023/0023122 A1
(43) Pub. Date: Jan. 26, 2023

(54) USE OF MORPHOMETRIC CHANGES IN THE BRAIN AS A BIOMARKER TO PREDICT BRAIN TUMOR SURVIVAL

(71) Applicant: Washington University, St. Louis, MO (US)

(72) Inventors: Eric Leuthardt, St. Louis, MO (US); Bidhan Lamichhane, St. Louis, MO (US)

(21) Appl. No.: 17/866,246

(22) Filed: Jul. 15, 2022

Related U.S. Application Data
(60) Provisional application No. 63/222,701, filed on Jul. 16, 2021.

(51) Int. Cl. A61B 5/00; A61B 5/107; A61B 5/055

(52) U.S. Cl. CPC A61B 5/7275; A61B 5/4064; A61B 5/1073; A61B 5/1072; A61B 5/0042; A61B 5/055

(57) ABSTRACT

The present disclosure is directed to methods of predicting overall survival, monitoring, and selecting treatments for a glioblastoma (GBM) patient. The method of the present disclosure includes obtaining at least one morphometric image from the GBM patient, identifying at least one radiomic biomarker based on the at least one morphometric image, and determining an overall survival value based on the at least one radiomic biomarker.

### CROSS-REFERENCE TO RELATED APPLICATIONS

This application claims the benefit of priority to U.S. Provisional Patent Application No. 63/222,701 filed Jul. 16, 2021, which is incorporated by reference herein in its entirety.

### STATEMENT REGARDING FEDERALLY SPONSORED RESEARCH & DEVELOPMENT

This invention was made with government support under R01 CA203861 awarded by the National Institutes of Health. The government has certain rights in the invention.

### FIELD OF THE DISCLOSURE

The field of the disclosure relates generally to predicting survival and selecting treatments in glioblastoma (GBM) patients.

### BACKGROUND OF THE DISCLOSURE

Glioblastoma multiforme (GBM) has poor survival with current treatments. Thus, there is a pressing need to identify biomarkers that improve pre-treatment planning and guide clinical trial protocols. Morphometric assessments of tumors and immediately adjacent areas have routinely used structural MRI. However, the existence and effects of distant structural changes from the tumor invasion site have been under-examined. Accordingly, there is a need for radiomic biomarkers that are easily accessible data at the time of diagnosis and that provide important prognostic information prior to surgery and/or treatment to help guide therapy.

### BRIEF DESCRIPTION OF THE DISCLOSURE

In one aspect, the present disclosure is directed to a method of predicting overall survival of a glioblastoma (GBM) patient. The method comprises obtaining at least one morphometric image from the GBM patient, identifying at least one radiomic biomarker based on the at least one morphometric image, and determining an overall survival value based on the at least one radiomic biomarker.

In another aspect, the present disclosure is directed to a method of monitoring a glioblastoma (GBM) patient. The method comprises obtaining at least one morphometric image from the GBM patient and identifying at least one radiomic biomarker based on the at least one morphometric image.

In yet another aspect, the present disclosure is directed to a method for selecting treatments for a glioblastoma (GBM) patient. The method comprises obtaining at least one morphometric image from the GBM patient, identifying at least one radiomic biomarker based on the at least one morphometric image, and selecting one or more treatments based on the at least one radiomic biomarker.

In some embodiments, the at least one radiomic biomarker comprises a structural change distant from a primary tumor mass, subcortical volume, and/or cortical thickness. In some embodiments, the at least one radiomic biomarker comprises right precuneus cortical thickness, temporal lobe cortical thickness, and/or occipital lobe cortical thickness.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The embodiments described herein may be better understood by referring to the following description in conjunction with the accompanying drawings.

FIG. 1 is an exemplary embodiment of heatmaps showing the distribution of tumor density (both left and right hemisphere) in accordance with the present disclosure. Defined by contrast-enhanced (CE) T1w boundaries, separately for GBM patients used in right and left hemispheric cortical thickness analysis.

FIG. 2(A-D) is an exemplary embodiment of visualization of differences in contralateral cortical thickness (overlaid on human connectome project's surface mesh, for visualization purpose) in accordance with the present disclosure. FIG. 2A shows a cortical map summary of parcel's cortical thickness of Healthy control (HC). FIG. 2B shows a cortical map summary of parcel's cortical thickness of GBM patients. FIG. 2C shows the differences in CT between groups (GBM-HC). The negative value (blue) in C represents the thinner cortex in GBM compared to HC (GBM<HC). FIG. 2D shows the plot of −log 10 of the p-value of the group difference in cortical thickness (showing only the parcels survived in multiple comparison corrections).

FIG. 3(A-D) is an exemplary embodiment of precuneus parcel visualization and overall survival in accordance with the present disclosure. FIG. 3A shows a visualization of precuneus parcel (blue) on human connectome project's surface mesh. FIG. 3B shows scatter plots of CT and OS (significant correlation between OS and right precuneus's CT (r=0.70, p<0.005, corrected). FIG. 3C shows overall survival in GBM patients with low CT (<median right precuneus's CT) differed significantly from overall survival in patients with high CT (>median right precuneus's CT) (Wilcoxon rank-sum, W=219 p<0.014, uncorrected). FIG. 3D shows Kaplan-Meier survival analysis comparing overall survival in low CT GBM patients and high CT patients (log-rank test, p=0.029, uncorrected). Patients with high CT had a significantly longer overall survival than those with low CT (HR: 0.59, 95% CI: 0.38-0.92,p=0.020).

### DETAILED DESCRIPTION OF THE DISCLOSURE

The prognosis of glioblastoma (GBM) remains poor. Recent findings have demonstrated prognostic value for widespread functional network anomalies that are incompletely explained by focal brain injury in the neighborhood of the tumor mass. As described herein, GBM also associates with widespread alterations of cortical thickness to provide prognostic value for overall survival. In particular, GBM patients demonstrated structural alterations distant from the tumor, morphometric changes were present at the time of diagnosis of GBM, and cortical thinning in select areas (right precuneus, temporal lobe, and occipital lobe) predicted overall survival in GBM.

The present disclosure is directed to methods of predicting survival in glioblastoma (GBM) patients based on measurements of cortical thickness and subcortical volumes obtained from morphometric images such as T1-weighted MR images. In particular, widespread cortical thinning in the contralateral hemisphere and right amygdala enlargement were present in GBM patients prior to treatment as compared to healthy controls. The pre-treatment cortical thickness of the right precuneus, temporal lobes, and occipital lobes had prognostic significance for the GBM patients.

### INTRODUCTION

One of the most malignant brain tumors is glioblastoma multiforme (GBM). Median overall survival prognosis (OS) is very poor, with death ranging from 12 to 15 months from diagnosis of GBM to death. An unmet clinical need is accessible, non-invasively acquired predictive biomarkers. Currently, structural Mill scans routinely provide morphometric radiologic assessments of the tumor; however, brain morphology distant from the tumor site has not influenced clinical prognosis, decisions of care or balancing aggressiveness of treatment with impacts on quality of life. Unfortunately, little is known about morphological changes in the brain separated from the tumor invasion site.

Prior studies reported associations between brain morphological changes and cognitive impairments in healthy aging and neurodegenerative diseases. GBM patients often present with neurologic deficits, typically attributed to tissue invasion and local anatomic mass effects. However, cognitive performance deficits are more severe in patients with high-than low-grade gliomas (HGG vs LGG), even after accounting for tumor volumes. More recently, motor deficits reported in pediatric patients with malignant glioma coincided with bilaterally thinner motor cortices, providing intriguing evidence of a relationship between impairment and more distant morphological changes. However, currently little is known regarding the effects of a focally destructive GBM on widespread structural changes.

Functional imaging provides further evidence of GBM effects on the whole brain. Specifically, brain functional organization, characterized in resting-state functional connectivity (rsFC), changed in GBM and similarly in several neurological diseases. Patients with GBM showed widespread distortions in the functional architecture beyond focally malignant tissue, and occurred bi-laterally. Notably, Stoecklein et al. found aberrant rsFC in the contralateral hemisphere associated with high-grade tumors.

Clinical and functional evidence suggest structural changes in GBM possibly have global effects not restricted to tumor sites. It was first determined whether there were brain morphological changes in GBM patients distinguishable from healthy controls. Furthermore, these changes were prognostic of patient OS. Specifically, cortical thickness (CT) was estimated in the hemisphere contralateral to the tumor site for de novo GBM patients and compared against CT from comparable cortical parcels in healthy controls. The findings supported the hypothesis of widespread morphological changes in GBM. These anatomically specific CT alterations may potentially serve as a prognostic biomarker aiding decisions for the care of GBM patients.

Materials and Methods

Cortical thickness (CT) and subcortical volumes were examined using FreeSurfer applied to high resolution T1-weighted structural images prior to standard treatment with surgery and chemoradiation in de novo GBM (20 right hemispheres, 30 left hemispheres, two bilateral hemispheres, 60.7 years mean patient age). FreeSurfer similarly processed CT in identical images in 24 healthy control (HC) subjects (60.3 years mean subject age). Changes were then studied in CT in GBM and correlations between such changes and overall survival.

Patients. GBM patients (N=79), retrospectively recruited from the neurosurgery brain tumor service at Washington University Medical Center, met the following criteria: newly diagnosed brain tumor, had surgical treatment of cancer, showed intracranial primary GBM pathology, and received a pre-surgical indication for structural MRI as determined by the treating neurosurgeon. Pathology identified by a neuropathologist in all cases met World Health Organization (WHO) and case specific histological criteria. Exclusion criteria were younger than 18 and prior surgery for a brain tumor. Table 1 lists patient demographics. We also studied structural data from normal healthy controls (HC) (N=24).

MRI Acquisition. Siemens Trio and Skyra Mill scanners, operating at 3T, provided structural MR images from each patient and HC using magnetization prepared rapid acquisition gradient echo (MPRAGE: TE=2.53 ms, TR=1900 ms, TI=900 ms, 256×256 acquisition matrix, 0.976×0.976×1 mm voxels) and T2-weighted fast spin-echo (FSE: TE=93 ms, TR=5600 ms, 256×256 acquisition matrix, 1.093×1.093×2 mm voxels).

FreeSurfer Segmentation. Visual inspections of all T1 and T2-weighted images ensured brain structures were free of blurring, ringing, striping, ghosting, etc., caused by head motion during scans. Freesurfer recon-all option (version 6.0) segmented T1w and T2w images. After initial processing, we removed gadolinium-enhanced and necrotic portions of the brain tumor from the resulting brain mask. We used tumor masks generated by the software application ITK-SNAP in a semi-automatic way, using multimodal images (T1w, postcontrast T1w, T2w, and FLAIR). This enabled separation of normal cortical and subcortical tissue from a contrast-enhancing tumor, necrosis, and surrounding FLAIR hyperintense edematous areas. A tumor (and mask) was defined as a contrast-enhancing plus necrotic-appearing region. Freesurfer “recon-all-all” ran on the tumor-masked brain mask and edited the resulting segmentations. We manually edited cases of inaccuracies by adding control points to help Freesurfer identify white matter (WM) voxels or by removing the skull and dura from the brain mask. Edema was not masked in the brain mask. Consequently, the reconstruction thresholded out edematous cortex in some patients. Manual patching did not address defects in surface topologies arising in the vicinity of the tumor. Freesurfer version 6.0 similarly processed T1w and T2w images in HCs but without applying a tumor mask.

A single rater (D. L. D.) reviewed segmentation in patients and controls to ensure data quality. Freesurfer segmentation failed after a week of processing in 19 of 79 GBM patients due to having a severe topological defect. Another patient was excluded due to immediate post-operative mortality. Retained data were from 59 patients. We further excluded 9 patients who had unrealizable CT-segmentation or had bilateral cortical tumors. Following exclusions, further cortical analysis was in 20 right and 30 left tumor patients.

Data Processing and Statistical Analysis Post-FreeSurfer. The post-Freesurfer analysis focused on 34 cortical parcels based on the Desikan-Killiany parcellation. Multiple regression models, ROI to ROI basis, then compared cortical thickness (CT) between GBM and HC groups, while controlling for age and sex. The R statistical language (R's linear model permutation function imp from the lmPerm package) executed all model fitting. Specifically, we estimated group difference, using effect coded group (i.e., HC=−1, GBM=1) as a categorical variable in the regression analysis and CT as a function of the group, age and sex (Model 1: CT group+age+sex). We used similar models to test the association between brain-morphological change and overall survival (OS), focusing on GBM-patients only. The linear model used for predicting OS by CT was tested as follows: OS˜CT+age+sex (Model 2).

A permutation test assessed statistical significance, using a total number of iterations=100,000 and the lmPerm package for R. Statistically significant results satisfied a p-value of the permutation (p)×34<0.05 (in other words, if original p is ⇐0.0014, equivalent to corrected p<0.05), where 34 is the total number of the possible test (34—contralateral cortical parcels).

Results

GBM patients compared with HC had widespread cortical thinning after correcting for age and sex. Cortical thinning in GBM patients occurred in occipital cortex, sensory cortex, right precuneus, right superior parietal areas, and right transverse temporal gyms. Cortical thickness in the right precuneus, temporal lobe, and occipital lobe predicted overall survival.

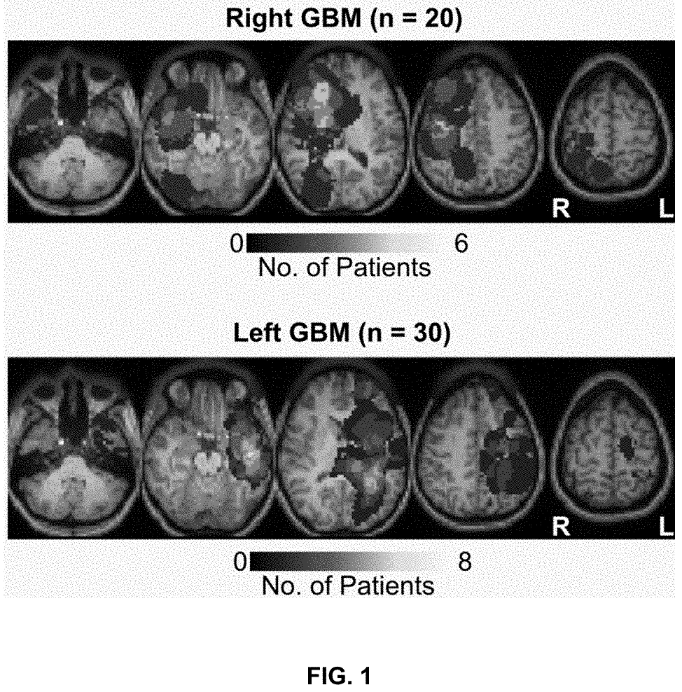

Study Samples. Table 1 lists the demographics of HC cases and assessed overall survival (OS) of the GBM cases. GBM patients were in two tumor groups: left or right hemisphere tumors. FIG. 1 illustrates the heterogeneity of GBM location, size, and morphology. The heat map indicates tumor density distribution in studied GBM patients, defined by contrast-enhanced (CE) T1w boundaries, segmented by using the software application ITK-SNAP.

TABLE 1
Demographic of the study sample and clinical information. HC = Healthy
control, GBM = Glioblastoma multiforme patients, OS = overall survival, LH = left-hemispheric
tumor, RH = right-hemispheric tumor, CE = contrast-enhanced bounded, IDH1 = isocitrate
dehydrogenase-1 R132, EGFR = epidermal growth factor receptor (EFGR) amplification, MGMT =
methylguanine-DNA methyltransferase promoter methylation.

| | Groups | | |
|---|---|---|---|
| | | GBM | |
| | | Tumor hemisphere | |
| Variables | HC | Right | Left |
| Patients (N) | 24 | 20 | 30 |
| Sex: Male | 12 | 14 | 21 |
| Female | 12 | 6 | 9 |
| Age (in years) | 60.33 | 66.10 | 55.36 |
| (range) | (54-66) | (53-83) | (22-83) |
| Mean/median OS (days) | — | 470.63/398 | 601/540 |
| (range) | | (111-1281) | (48-1801) |
| Missing | | 4-patients | 4-patients |
| CE volume (cm³) | — | 31.94 ± 39.79 | 23.93 ± 26.47 |
| IDH1: Mutated | — | 1 | 2 |
| Wild type | | 19 | 27 |
| Missing | | 0 | 1 |
| MGMT: Methylated | | 9 | 10 |
| Non-methylated | — | 10 | 16 |
| Missing | | 1 | 4 |
| EGFR: Positive | | 7 | 8 |
| Negative | — | 7 | 9 |
| Missing | | 6 | 13 |

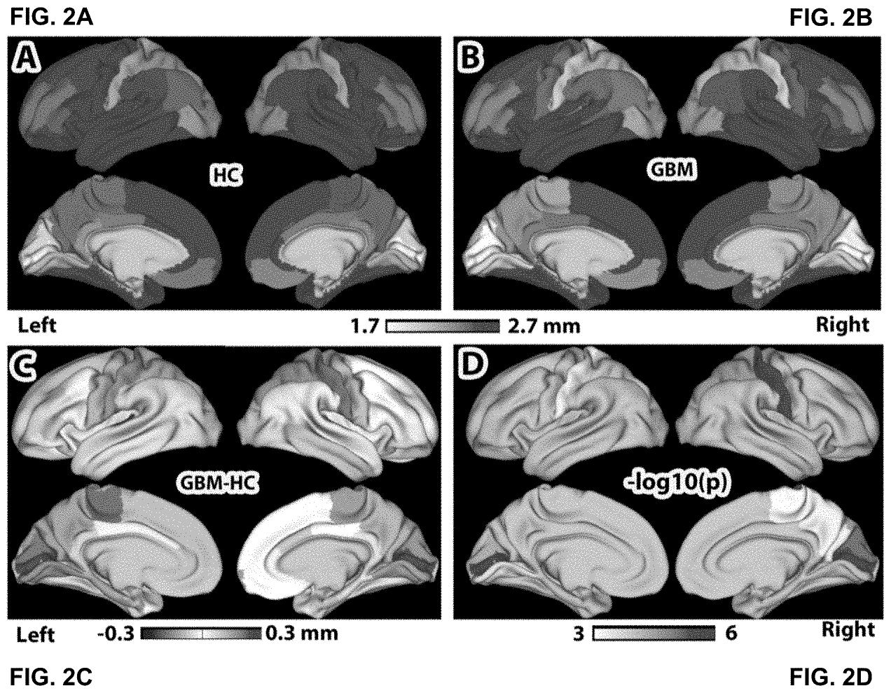

Differences of Cortical Thickness (CT). Table 2 lists mean (±standard deviation) of CT parcels with significant group differences (if multiple comparisons corrected p<0.05 and using permutation resampling method), and sp-value of significance in multiple linear regression models (Model 1). GBM patients had thinner cortices in eight out of 34 cortical parcels in the right hemisphere (left tumor patients): cuneus, lingual gyms of the occipital lobe, paracentral gyms, pericalcarine cortex, postcentral gyms, precuneus, superior-parietal cortex, and transverse temporal gyms. Similarly, in the left hemisphere (right tumor patients) GBM patients showed thinning in four parcels: cuneus, lingual gyms, pericalcarine cortex, postcentral gyms. FIG. 2(A-D) shows plots of measured CT in HCs (FIG. 2A), GBMs (FIG. 2B), group differences in CT (GBM<HC) (FIG. 2C), and the −log 10 of p-value of group difference in the multiple regression analysis (FIG. V2D) (Model-1, after correcting for age and sex, separately in left and right hemisphere parcels). Parcels marked in blue had thinner CT in GBM. GBM compared to HC cases showed no significantly increased cortical thickness (see supplemental for details).

TABLE 2
Parcel-wise cortical thickness of GBM and HC. Mean CT ( ± sd) of
cortical parcels, p-value of group difference (GBM vs HC) in multiple linear models (Model 1) of
significant parcels (if p<0.05 after multiple comparisons and using permutation resampling). * = p
< 0.05 (after Bonferroni correction).

| | Mean CT ± SD (mm) | | |
|---|---|---|---|
| Cortical structure | Healthy | GBM | p-value |
| (parcel name) | controls | patients | (uncorrected) |

Right hemispheric CT (Tumor on left hemisphere, N = 30)

| | | | |
|---|---|---|---|
| Cuneus | 1.98 ± 0.12 | 1.81 ± 0.12 | 1.00E−16* |
| Lingual | 2.11 ± 0.13 | 1.97 ± 0.13 | 8.00E−05* |
| Paracentral | 2.42 ± 0.11 | 2.27 ± 0.11 | 0.00045* |
| Pericalcarine | 1.75 ± 0.15 | 1.49 ± 0.15 | 1.00E−16* |
| Postcentral | 2.14 ± 0.08 | 1.94 ± 0.08 | 1.00E−16* |
| Precuneus | 2.39 ± 0.08 | 2.27 ± 0.08 | 0.00043* |
| Superior-parietal | 2.25 ± 0.10 | 2.08 ± 0.10 | 1.00E−16* |
| Transverse-temporal | 2.39 ± 0.16 | 2.19 ± 0.16 | 9.00E−05* |

Left hemispheric CT (Tumor on right hemisphere, N = 20)

| | | | |
|---|---|---|---|
| Cuneus | 1.95 ± 0.15 | 1.75 ± 0.11 | 1.00E−05* |
| Lingual | 2.09 ± 0.11 | 1.92 ± 0.11 | 7.00E−05* |
| Pericalcarine | 1.74 ± 0.13 | 1.48 ± 0.09 | 1.00E−16* |
| Postcentral | 2.13 ± 0.09 | 1.97 ± 0.15 | 0.00041* |

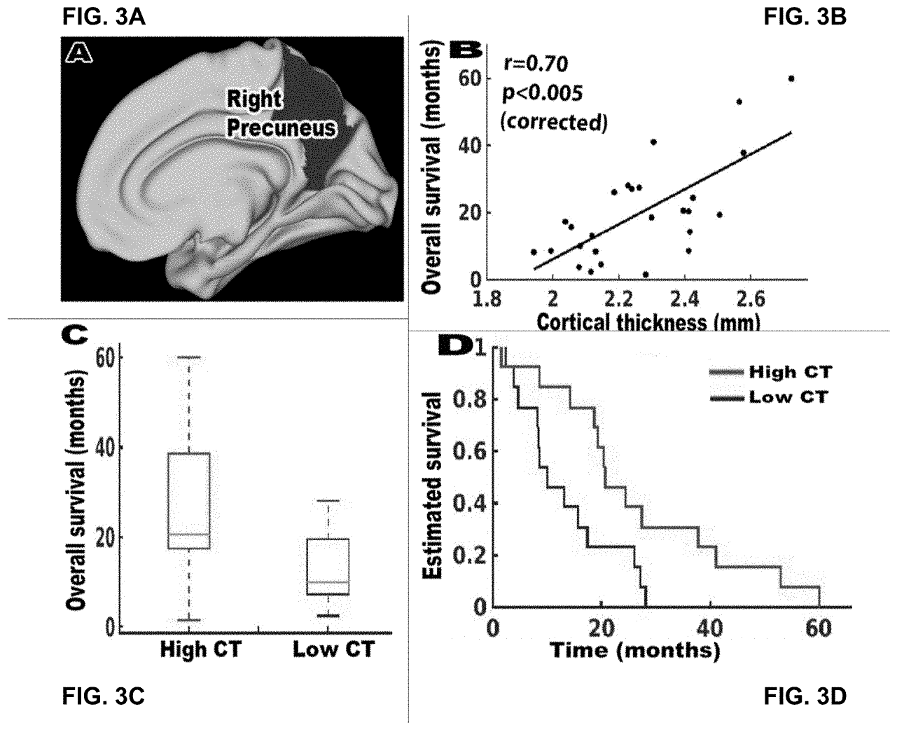

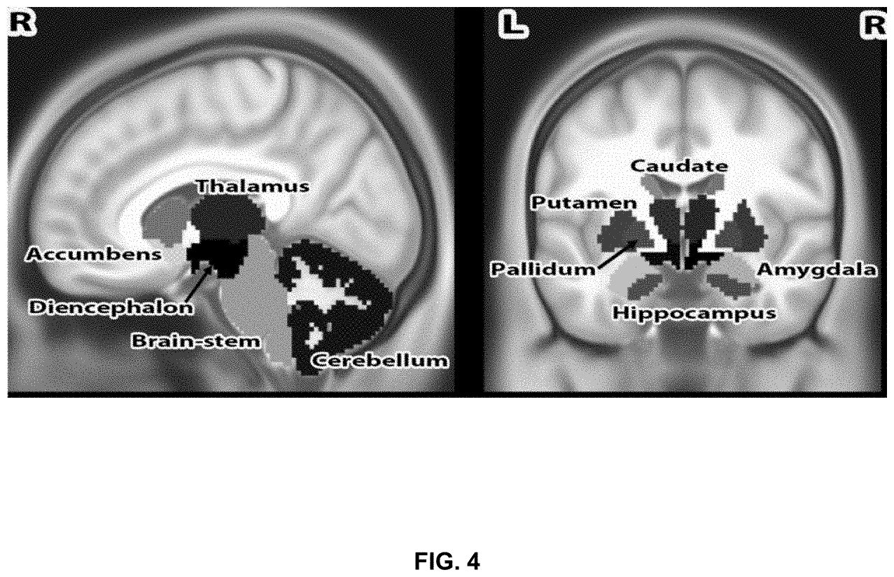

Association Between Brain-morphological Change and Overall Survival. The examined relationship between CT in individual parcels and OS was done separately for left and right hemisphere parcels, using multiple regression models (Model 2: OS˜CT+age+sex), controlled for age and sex. Notably, the CT of the right precuneus (of patients with tumor in the left hemisphere) was found to be a significant predictor of the OS (beta=0.685, p<0.0006 (uncorrected and equivalent to corrected p<0.05). No other parcels showed a significant association between CT and OS (all p>0.05, after multiple comparison correction) (see supplement for details). The association of right precuneus CT and OS was supported by a significant correlation between right precuneus CT and OS (r=0.70, p<0.005, corrected, FIG. 3B).

A further Kaplan-Meier survival analysis was of the association between cortical thinning in the right precuneus and OS. For this, the patients were median split into low CT (<median right precuneus's CT) and high CT groups. Median OS in the high CT group (20.67 months) was significantly longer than that in the low CT group (10.03 months) (right-tailed Wilcoxon rank-sum, W=219, p<0.014, uncorrected) (FIG. 3C). Kaplan-Meier analysis also demonstrated a significant difference in OS between Low and high CT groups (log-rank test, p=0.029) (FIG. 3D). Also, the Cox proportional hazard model suggested a significantly longer OS of those with high than low CT (Hazard ratio (HR): 0.59, 95% CI: 0.38-0.92, p=0.02).

We computed multivariate Cox regressions, controlling clinical and demographic covariates on survival times (Table 3). The univariate Cox regression, showed cortical thickness was a significant predictor of survival (HR: 0.37, CI: 0.21, 0.65, p=0.0007), and this effect was maintained with the inclusion of age and tumor size as covariates (HR: 0.37, CI: 0.21, 0.68, p=0.0013).

Relationship between lobar cortical thickness and overall survival. In addition to the parcels-based analysis per hemisphere, lobe-specific mean cortical thickness over both hemispheres also correlated between lobes (frontal, parietal, temporal, occipital, and cingulate, a total of 5 lobes defined by Freesurfer) and OS. After Bonferroni correction (with correction factor 5), the temporal (r=0.45, p<0.0026) and occipital (r=0.43, p<0.0044) lobes significantly correlated with OS (complete analysis of the association between OS and lobe-specific cortical thickness was presented in supplementary Table 8). Further, a univariate regression analysis confirmed the same effect: CT of both the temporal lobe (HR: 0.71, CI: 0.55, 0.93, p=0.013) and occipital lobe (HR: 0.61, CI: 0.43, 0.88, p=0.008) were significant predictors of OS (Table3). In Multivariate Cox regressions analysis, controlling clinical and demographic covariates on survival times, the lobar CT of the occipital lobe remained a significant predictor of OS (HR: 0.60, CI: 0.41, 0.89, p=0.012) and trended similarly toward significance in the temporal lobe (HR: 0.71, CI: 0.48, 1.01, p=0.06) (Table 3).

TABLE 3
Survival analysis. Cox proportional hazards
model was performed for
univariate and multivariate regression
(with age and tumor size as covariates).

| | | | Multivariate Cox | |
|---|---|---|---|---|
| | | | (age and tumor | |
| | | | size were as | |
| | Univariate Cox | | covariates) | |
| Characteristic | HR (95% CI) | P | HR (95% CI) | P |

Right Precuneus

| | | | | |
|---|---|---|---|---|
| Age at initial diagnosis | 1.32 (0.74, 2.34) | 0.330 | 1.00 (0.55,1.81) | 0.99 |
| Tumor volume (cm³) | 1.38 (.99, 1.94) | 0.058 | 1.41 (.94, 2.10) | 0.094 |
| Right Precuneus CT | 0.37 (0.21, 0.65) | 0.0007 | 0.37 (0.21, 0.68) | 0.0013 |

Temporal lobe

| | | | | |
|---|---|---|---|---|
| Age at initial diagnosis | 1.23 (0.86, 1.79) | 0.255 | 0.97 (0.60, 1.56) | 0.92 |
| Tumor volume (cm³) | 1.26 (0.98,1.94) | 0.072 | 1.25 (0.96, 1.66) | 0.10 |
| Temporal lobe CT | 0.71 (0.55,0.93) | 0.013 | 0.71 (.48 1.01) | 0.06 |

Occipital lobe

| | | | | |
|---|---|---|---|---|
| Age at initial diagnosis | 1.23 (0.86, 1.79) | 0.255 | 1.02 (0.67,1.53) | 0.94 |
| Tumor volume (cm³) | 1.26 (0.98,1.94) | 0.072 | 1.36 (1.02,1.82) | 0.038 |
| Occipital lobe CT | 0.61 (0.43,0.88) | 0.008 | 0.60 (0.41,0.89) | 0.012 |

Subcortical Volume (SV). Subcortical volume analysis was focused on 19 subcortical regions of interest (ROIs) that were anatomically defined by Freesurfer (FIG. 4, for visualization purpose).

Group Differences of Subcortical Volume (SV). The mean±standard deviation (sd) of Freesurfer segmented volumes of all 19 areas of interest were reported in Table 4. Similarly, the beta parameter (weight) of group difference in multiple linear models after regressing out the effect of age and sex and the p-value of significance using permutation were also reported in Table 4.

TABLE 4
The comparisons of gray matter volumes in the subcortical regions
between patients with GBM and healthy controls. The mean volume ± sd, the beta parameter
(effect) of group difference in multiple linear model (Model 1) of each subcortical ROI are
reported. (*/bold = p < 0.05, permutation resampling and Bonferroni correction).

| | Mean Volume ± SD (mm³) | | Beta | |
|---|---|---|---|---|
| Subcortical regions | Healthy controls | GBM | (weight) | p-value |
| 1. Left Accumbens | 505.63 ± 79.19 | 490.98 ± l 17.34 | −0.089 | 0.47617 |
| 2. Right Accumbens | 497.05 ± 68.05 | 508.69 ± 91.67 | 0.045 | 0.69092 |
| 3. Left Amygdala | 1557.77 ± 230.75 | 1723.05 ± 377.61 | 0.208 | 0.09433 |
| 4. Right Amygdala | 1589.50 ± 204.56 | 1919.93 ± 422.54 | 0.394 | 0.00021* |
| 5. Brain-Stem | 21810.53 ± 2810.82 | 21656.78 ± 2486.05 | −0.052 | 0.68679 |
| 6. Left Caudate | 3356.39 ± 373.26 | 3120.38 ± 405.53 | −0.312 | 0.01769 |
| 7. Right Caudate | 3438.40 ± 373.78 | 3289.74 ± 576.85 | −0.170 | 0.20143 |
| 8. Left Hippocampus | 3913.98 ± 437.80 | 4082.13 ± 819.45 | 0.081 | 0.51802 |
| 9. Right Hippocampus | 4004.67 ± 397.86 | 4235.47 ± 518.07 | 0.207 | 0.08312 |
| 10. Left Pallidum | 1973.09 ± 224.45 | 1981.60 ± 286.08 | 0.000 | 1 |
| 11. Right Pallidum | 1806.28 ± 210.84 | 1989.94 ± 302.51 | 0.308 | 0.01546 |
| 12. Left Putamen | 4694.429 ± 498.16 | 4729.38 ± 796.15 | 0.012 | 0.9136 |
| 13. Right Putamen | 4583.96 ± 467.60 | 4627.64 ± 633.42 | 0.015 | 0.88774 |
| 14. Left Thalamus | 7226.53 ± 769.80 | 7217.62 ± 1216.66 | −0.036 | 0.75044 |
| 15. Right Thalamus | 7225.12 ± 795.50 | 6898.73 ± 983.00 | −0.193 | 0.08278 |
| 16. Left Cerebellum. | 53864.28 ± 5462.38 | 55515.86 ± 6514.48 | 0.119 | 0.3411 |
| 17. Right Cerebellum. | 54798.08 ± 5672.35 | 55708.09 ± 6133 39 | 0.054 | 0.67245 |
| 18. Left Diencephalon | 4020.40 ± 461.42 | 4165.87 ± 566.56 | 0.106 | 0.3785 |
| 19.Right Diencephalon | 3919.62 ± 412.56 | 4012.18 ± 480.33 | 0.072 | 0.55585 |

Association Between SV Changes and Overall Survival. The relationship of the SV with OS was tested using multiple regression models (Model 2) while controlling for age and sex. SV was modeled as a function of the group, age, and sex. No significant association was found between SV and OS (for all subcortical ROIs, p>0.09 (uncorrected). The beta parameter (weights) of association between SV and OS in multiple linear models after regressing out the effect of age and sex and the p-value of significance using permutation were also reported in Table 5.

TABLE 5
The relationship between SV and OS (OS prediction). The beta parameter
(using Model 2) and p-value of the complete set of analyses.

| | Beta | |
|---|---|---|
| Subcortical regions | (weight) | p-value |
| 1. Left Accumbens | 0.1735507 | 0.39241 |
| 2. Right Accumbens | 0.181061 | 0.41956 |
| 3. Left Amygdala | 0.0954599 | 0.64807 |
| 4. Right Amygdala | 0.0290939 | 0.88452 |
| 5. Brain-Stem | 0.3394866 | 1 |
| 6. Left Caudate | 0.0913897 | 0.62535 |
| 7. Right Caudate | 0.3172283 | 0.08985 |
| 8. Left Hippocampus | 0.1945043 | 0.34469 |
| 9. Right Hippocampus | 0.1608019 | 0.45139 |
| 10. Left Pallidum | 0.2545303 | 0.19068 |
| 11. Right Pallidum | 0.2726461 | 0.14376 |
| 12. Left Putamen | 0.0045847 | 1 |
| 13. Right Putamen | 0.0758292 | 0.74999 |
| 14. Left Thalamus | 0.1992525 | 0.38291 |
| 15. Right Thalamus | 0.2595413 | 0.25309 |
| 16. Left Cerebellum. | 0.225092 | 1 |
| 17. Right Cerebellum. | 0.2291357 | 1 |
| 18. Left Diencephalon | 0.30979 | 0.23777 |
| 19.Right Diencephalon | 0.296346 | 0.20002 |

Cortical Thickness (CT). Cortical thickness analysis was focused on 34 parcels of interest of the right hemisphere (of left tumor subjects) and left hemisphere (of the right tumor subject) that were anatomically defined using Desikan-Killiany parcellation in Freesurfer (as discussed in the main text).

Group differences (HC Vs. GBM) in the right hemispheric cortical thickness. The mean±standard deviation (sd) of CT of all parcels of interest are reported in Table 6. For the statistical inference, the beta parameter (effect) of group difference in multiple linear model after regressing out the effect of age and sex, and the p-value of significance using permutation test were also reported.

The comparisons of cortical thickness between GBM and HC. The mean
CT ± sd of cortical parcels, the beta parameter (effect) of group difference in multiple linear model
(Model 1) per cortical parcel were presented in the table. (*/bold=p < 0.05, permutation resampling
and Bonferroni correction).

| | Mean CT ± SD (mm) | | | |
|---|---|---|---|---|
| Cortical structure | Healthy | | Beta | |
| (parcel name) | controls | GBM | (weight) | p-value |
| 1. Banks-sts | 2.49 ± 0.12 | 2.61 ± 0.12 | 0.210 | 0.1113 |
| 2. Caudal-anteriorcingulate | 2.33 ± 0.13 | 2.43 ± 0.13 | 0.284 | 0.04648 |
| 3. C audal -mi ddl efrontal | 2.46 ± 0.12 | 2.46 ± 0.12 | −0.067 | 0.638 |
| 4. Cuneus | 1.98 ± 0.12 | 1.81 ± 0.12 | −0.606 | 1.00E−16* |
| 5. Entorhinal | 3.47 ± 0.25 | 3.39 ± 0.25 | −0.099 | 0.50221 |
| 6. Fusiform | 2.71 ± 0.09 | 2.65 ± 0.09 | −0.315 | 0.01713 |
| 7. Inferior-parietal | 2.46 ± 0.09 | 2.42 ± 0.08 | −0.267 | 0.04538 |
| 8. Inferior-temporal | 2.69 ± 0.12 | 2.80 ± 0.12 | 0.234 | 0.06685 |
| 9. Isthmus-cingulate | 2.27 ± 0.13 | 2.33 ± 0.13 | 0.128 | 0.38307 |
| 10. Lateral-occipital | 2.28 ± 0.09 | 2.20 ± 0.09 | −0.401 | 0.00281 |
| 11. Lateral-orbitofrontal | 2.47 ± 0.09 | 2.57 ± 0.09 | 0.230 | 0.08025 |
| 12. Lingual | 2.11 ± 0.13 | 1.97 ± 0.13 | −0.536 | 8.00E−05* |
| 13. Medial-orbitofrontal | 2.29 ± 0.11 | 2.30 ± 0.11 | −0.149 | 0.18123 |
| 14. Middle-temporal | 2.78 ± 0.10 | 2.89 ± 0.10 | 0.327 | 0.00778 |
| 15. Parahippocampal | 2.70 ± 0.22 | 2.65 ± 0.22 | −0.156 | 0.28648 |
| 16. Paracentral | 2.42 ± 0.11 | 2.27 ± 0.11 | −0.457 | 0.00045* |
| 17. Parsopercularis | 2.51 ± 0.11 | 2.52 ± 0.11 | −0.047 | 0.73172 |
| 18. Parsorbitalis | 2.58 ± 0.16 | 2.66 ± 0.16 | 0.024 | 0.83779 |
| 19. Parstriangularis | 2.37 ± 0.09 | 2.41 ± 0.09 | 0.057 | 0.66516 |
| 20. Pericalcarine | 1.75 ± 0.15 | 1.49 ± 0.15 | −0.744 | 1.00E−16* |
| 21. Postcentral | 2.14 ± 0.08 | 1.94 ± 0.08 | −0.683 | 1.00E−16* |
| 22. Posterior-cingulate | 2.34 ± 0.11 | 2.33 ± 0.11 | −0.087 | 0.54156 |
| 23. Precentral | 2.49 ± 0.13 | 2.39 ± 0.13 | −0.328 | 0.01964 |
| 24. Precuneus | 2.39 ± 0.08 | 2.27 ± 0.08 | −0.466 | 0.00043* |
| 25. Rostral anteriorcingulate | 2.70 ± 0.18 | 2.74 ± 0.18 | 0.051 | 0.71554 |
| 26. Rostral-middlefrontal | 2.32 ± 0.11 | 2.30 ± 0.11 | −0.182 | 0.17322 |
| 27. Superior-frontal | 2.58 ± 0.09 | 2.59 ± 0.09 | −0.090 | 0.49132 |
| 28. Superior-parietal | 2.25 ± 0.10 | 2.08 ± 0.10 | −0.580 | 1.00E−16* |
| 29. Superior-temporal | 2.74 ± 0.11 | 2.77 ± 0.11 | −0.095 | 0.32366 |
| 30. Supramarginal | 2.49 ± 0.07 | 2.46 ± 0.07 | −0.214 | 0.11437 |
| 31. Frontal-pole | 2.56 ± 0.17 | 2.75 ± 0.17 | 0.297 | 0.02531 |
| 32. Temporal-pole | 3.61 ± 0.27 | 3.65 ± 0.27 | 0.021 | 0.88315 |
| 33. Transverse-temporal | 2.39 ± 0.16 | 2.19 ± 0.16 | −0.473 | 9.00E-05* |
| 34. Insula | 2.87 ± 0.17 | 2.90 ± 0.17 | −0.026 | 0.83931 |

Association between right-hemispheric CT and Overall Survival. The relationship of the CT with OS was tested using multiple regression models (Model 2) while controlling for age and sex, and complete results were listed in table 7.

TABLE 7
Overall survival (OS) prediction using right
cortical thickness (left tumor
subjects) in the general linear model when regressing
out the age and sex effect. (*/bold = p < 0.05,
corrected).

| Cortical structure | Beta | |
|---|---|---|
| (parcel name) | (weight) | p-value |
| 1. Banks-sts | 0.140 | 0.54436 |
| 2. Caudal-anteriorcingulate | −0.157 | 0.47478 |
| 3. Caudal-middlefrontal | 0.366 | 0.07853 |
| 4. Cuneus | 0.119 | 0.61478 |
| 5. Entorhinal | 0.297 | 0.18489 |
| 6. Fusiform | 0.543 | 0.02098 |
| 7. Inferior-parietal | 0.443 | 0.05094 |
| 8. Inferior-temporal | 0.350 | 0.14949 |
| 9. Isthmus-cingulate | 0.262 | 0.19626 |
| 10. Lateral-occipital | 0.583 | 0.01456 |
| 11. Lateral-orbitofrontal | 0.401 | 0.05506 |
| 12. Lingual | 0.402 | 0.06844 |
| 13. Medial-orbitofrontal | 0.506 | 0.05803 |
| 14. Middle-temporal | 0.415 | 0.09296 |
| 15. Parahippocampal | 0.274 | 0.16987 |
| 16. Paracentral | 0.382 | 0.07762 |
| 17. Parsopercularis | 0.177 | 0.46638 |
| 18. Parsorbitalis | 0.391 | 0.1011 |
| 19. Parstriangularis | −0.016 | 0.94077 |
| 20. Pericalcarine | 0.339 | 0.12426 |
| 21. Postcentral | 0.407 | 0.04074 |
| 22. Posterior-cingulate | 0.199 | 0.34223 |
| 23. Precentral | 0.308 | 0.12345 |
| 24. Precuneus | 0.685 | 0.00059* |
| 25. Rostral-anteriorcingulate | −0.107 | 0.60615 |
| 26. Rostral-middlefrontal | 0.355 | 0.10964 |
| 27. Superiorfrontal | 0.425 | 0.05419 |
| 28. Superior-parietal | 0.416 | 0.0506 |
| 29. Superior-temporal | 0.644 | 0.07876 |
| 30. Supramarginal | 0.518 | 0.01085 |
| 31. Frontal-pole | 0.238 | 0.25698 |
| 32. Temporal-pole | 0.447 | 0.03335 |
| 33. Transverse-temporal | 0.325 | 0.17147 |
| 34. Insula | 0.335 | 0.13474 |

Group differences (HC Vs. GBM) in the left hemispheric cortical thickness. The mean±standard deviation (sd) of left hemispheric CT of all parcels of interest were reported in Table 8.

TABLE 8
The comparisons of left-hemispheric cortical thickness between GBM
and HC. The mean CT ± sd of cortical parcels, the beta parameter (effect) of group difference in
multiple linear model (Model 1) per cortical parcel were presented in the table. (*/bold = p <
0.05, permutation resampling and Bonferroni correction).

| | Mean CT ± | | | |
|---|---|---|---|---|
| Cortical structure | Healthy | SD (mm) | Beta | |
| (parcel name) | controls | GBM | (weight) | p-value |
| 1. Banks-sts | 2.45 ± 0.12 | 2.40 ± 0.19 | −0.006 | 1 |
| 2. Caudal-anteriorcingulate | 2.56 ± 0.17 | 2.59 ± 0.21 | 0.098 | 0.56301 |
| 3. Caudal-middlefrontal | 2.46 ± 0.14 | 2.44 ± 0.17 | 0.025 | 1 |
| 4. Cuneus | 1.95 ± 0.15 | 1.75 ± 0.11 | −0.623 | 1.00E−05* |
| 5. Entorhinal | 3.36 ± 0.28 | 3.19 ± 0.27 | −0.163 | 0.30615 |
| 6. Fusiform | 2.67 ± 0.09 | 2.59 ± 0.20 | −0.009 | 0.95056 |
| 7. Inferior-parietal | 2.39 ± 0.09 | 2.34 ± 0.19 | −0.008 | 1 |
| 8. Inferior-temporal | 2.71 ± 0.13 | 2.77 ± 0.17 | 0.337 | 0.03537 |
| 9. Isthmus-cingulate | 2.23 ± 0.12 | 2.32 ± 0.22 | 0.374 | 0.02067 |
| 10. Lateral-occipital | 2.22 ± 0.12 | 2.16 ± 0.13 | −0.132 | 0.40751 |
| 11. Lateral-orbitofrontal | 2.53 ± 0.12 | 2.55 ± 0.18 | 0.204 | 0.1926 |
| 12. Lingual | 2.09 ± 0.11 | 1.92 ± 0.11 | −0.500 | 7.00E−05* |
| 13. Medial-orbitofrontal | 2.35 ± 0.16 | 2.26 ± 0.22 | −0.080 | 0.58754 |
| 14. Middle-temporal | 2.75 ± 0.12 | 2.80 ± 0.21 | 0.315 | 0.04404 |
| 15. Parahippocampal | 2.73 ± 0.25 | 2.60 ± 0.31 | −0.152 | 0.31728 |
| 16. Paracentral | 2.37 ± 0.11 | 2.15 ± 0.22 | −0.415 | 0.00282 |
| 17. Parsopercularis | 2.50 ± 0.11 | 2.47 ± 0.18 | 0.070 | 0.63494 |
| 18. Parsorbitalis | 2.58 ± 0.17 | 2.55 ± 0.18 | −0.057 | 0.72363 |
| 19. Parstriangularis | 2.41 ± 0.10 | 2.32 ± 0.20 | −0.113 | 0.42447 |
| 20. Pericalcarine | 1.74 ± 0.13 | 1.48 ± 0.10 | −0.734 | 1.00E−16* |
| 21. Postcentral | 2.13 ± 0.09 | 1.97 ± 0.15 | −0.521 | 0.00041* |
| 22. Posterior-cingulate | 2.33 ± 0.10 | 2.36 ± 0.20 | 0.233 | 0.14625 |
| 23. Precentral | 2.49 ± 0.14 | 2.37 ± 0.19 | −0.260 | 0.10056 |
| 24. Precuneus | 2.37 ± 0.11 | 2.23 ± 0.22 | −0.244 | 0.10105 |
| 25. Rostral anteriorcingulate | 2.69 ± 0.23 | 2.57 ± 0.26 | −0.127 | 0.43283 |
| 26. Rostral-middlefrontal | 2.35 ± 0.10 | 2.28 ± 0.17 | −0.101 | 1 |
| 27. Superior-frontal | 2.63 ± 0.11 | 2.25 ± 0.16 | −0.240 | 0.11012 |
| 28. Superior-parietal | 2.26 ± 0.09 | 2.12 ± 0.18 | −0.412 | 0.00623 |
| 29. Superior-temporal | 2.69 ± 0.13 | 2.63 ± 0.24 | −0.024 | 0.87568 |
| 30. Supramarginal | 2.45 ± 0.09 | 2.37 ± 0.14 | −0.154 | 0.31774 |
| 31. Frontal-pole | 2.67 ± 0.20 | 2.58 ± 0.33 | −0.166 | 0.33508 |
| 32. Temporal-pole | 3.51 ± 0.26 | 3.51 ± 0.35 | 0.096 | 0.55536 |
| 33. Transverse-temporal | 2.37 ± 0.19 | 2.05 ± 0.31 | −0.476 | 0.00175 |
| 34. Insula | 2.84 ± 0.14 | 2.79 ± 0.21 | 0.050 | 0.74696 |

Association between left-hemispheric CT and Overall Survival. In the test of the relationship of the CT with OS, using multiple regression models (Model 2) while controlling for age and sex, none of the left hemispheric parcels survived after Bonferroni correction. The complete set of analytics was reported in table 9.

TABLE 9
Overall survival (OS) prediction using left
cortical thickness (right
subjects) in the general linear model when
regressing out the age and sex effect.

| Cortical structure | Beta | |
|---|---|---|
| (parcel name) | (weight) | p-value |
| 1. Banks-sts | −0.1483162 | 0.63379 |
| 2. Caudal-anteriorcingulate | 0.33068289 | 0.31684 |
| 3. Caudal-middlefrontal | −0.1736753 | 0.55455 |
| 4. Cuneus | −0.1070959 | 0.7198 |
| 5. Entorhinal | 0.08043849 | 0.78697 |
| 6. Fusiform | 0.14133316 | 0.70406 |
| 7. Inferior-parietal | −0.3123436 | 0.38341 |
| 8. Inferior-temporal | −0.0490034 | 1 |
| 9. Isthmus-cingulate | 0.3082637 | 0.36386 |
| 10. Lateral-occipital | 0.02562131 | 0.93672 |
| 11. Lateral-orbitofrontal | −0.0293206 | 0.92226 |
| 12. Lingual | 0.25222211 | 0.44617 |
| 13. Medial-orbitofrontal | −0.3576412 | 0.36735 |
| 14. Middle-temporal | 0.09084395 | 0.77588 |
| 15. Parahippocampal | 0.3127891 | 0.31854 |
| 16. Paracentral | −0.2254532 | 0.49777 |
| 17. Parsopercularis | 0.31424039 | 0.4176 |
| 18. Parsorbitalis | 0.13985993 | 0.65097 |
| 19. Parstriangularis | 0.01929848 | 0.96188 |
| 20. Pericalcarine | −0.0669533 | 0.82021 |
| 21. Postcentral | −0.235825 | 0.47685 |
| 22. Posterior-cingulate | 0.71978647 | 0.01875 |
| 23. Precentral | −0.2675668 | 0.38311 |
| 24. Precuneus | 0.1817309 | 0.59707 |
| 25. Rostral-anteriorcingulate | 0.17433628 | 0.57687 |
| 26. Rostral-middlefrontal | −0.1583195 | 0.61711 |
| 27. Superiorfrontal | −0.3664962 | 0.26856 |
| 28. Superior-parietal | −0.419006 | 0.24778 |
| 29. Superior-temporal | 0.18524181 | 0.52998 |
| 30. Supramarginal | −0.347036 | 0.28663 |
| 31. Frontal-pole | −0.1181701 | 0.68277 |
| 32. Temporal-pole | −0.0053752 | 0.986 |
| 33. Transverse-temporal | −0.0153006 | 0.96128 |
| 34. Insula | 0.06016798 | 0.85195 |

Relationship between lobar cortical thickness and overall survival. The correlation between the lobe-specific measured value of cortical thickness, mean over bilateral lobes (frontal, parietal, temporal, occipital, and cingulate, a total of 5 lobes defined by Freesurfer) were shown in Table 10.

TABLE 10
Correlation between mean CT, mean
over lobes (both left and right
hemispheric parcels), and OS. */bold = p < 0.05
(corrected, using multiple correction factor = 5).

| | Combine left and right | |
|---|---|---|
| Name of the lobe | r | P |
| Frontal | 0.34 | 0.0273 |
| Parietal | 0.39 | 0.0115 |
| Temporal | 0.45 | 0.0026* |
| Occipital | 0.43 | 0.0044* |
| Cingulate | 0.15 | 0.3564 |

### DISCUSSION

No prior study examined contralesional cortical thickness in GBM patients and the association between CT with clinical outcomes. We found GBM patients had diffuse, distant changes in brain morphology at the time of diagnosis located separately from the tumor. Furthermore, cortical thinning in the right precuneus strongly correlated with overall survival. Findings of widespread structural alterations associated with focal glioblastomas, easily identified with MM imaging, might potentially serve as prognostic biomarkers.

Widespread Reduction in Cortical Thickness in GBM Cortical areas showing significant thinning were regions previously associated with higher-order multisensory and cognitive processing (i.e., R. Precuneus, R. superior-parietal), motor processing (R. Paracentral), and sensory functions (Somatosensory: R/L. postcentral Auditory: R. Transverse-temporal; Occipital and higher-order visual areas, R/L. Pericalcarine, R/L. Cuneus, R/L. Lingual). Mechanisms possibly responsible for a significant correlation between reductions in cortical thickness and presence of GBM, are unknown. Several possible hypotheses include: (1) a rapidly growing tumor that parasitizes nutrients, leading to cortical atrophy in metabolically and synthetically active brain regions; (2) a locally destructive tumor altering distant connectivity and synaptic homeostasis, resulting in reduced input and subsequent diminution in distant cortical sites; and (3) cortical changes at the time of GBM diagnosis, preceding oncogenesis and rather reflecting brain health generally, predisposing tumor development. Further study, however, will be needed given the hypothesized diverse mechanisms underpinning GBM and associated cortical thickness changes.

Cortical Thickness Predicts OS in GBM Thinner cortical thickness in GBM than HC in the right precuneus, and temporal and occipital lobes, showed a significant association with OS after Bonferroni correction. Specifically, GBM patients with thinner cortex had shorter overall survival.

Notably, the precuneus has a high resting metabolic rate, consuming ˜35% more glucose than any other area in the cerebral cortex in humans; and it is one of the hub regions known to be highly connected (for review, Cavanna and Trimble, 2006). As a metabolic precedent, patients with anorexia nervosa, a psychiatric disorder characterized by a restriction of food intake, showed cortical thinning in the right precuneus, which was then found correlated with the nutritional state as well as cognitive functions. Functionally, hypometabolism in this area has been reported in patients with cognitive decline (e.g., memory, language, and executive function) associated with different subtypes of dementia. Although the neurobiology of widespread cortical thinning particular to GBM remains unclear, cortical thinning might be a marker of disease severity (both metabolic and/or functional), affecting overall survival.

### CONCLUSION

The findings described in the present disclosure identified previously unnoticed brain structural changes distant from the primary tumor mass. These changes have prognostic information and may be valuable for treatment planning. Disclosed herein is the first recognition of cortical thickness as a prognostic biomarker for GBM.

Patients with GBM have multiple regions with cortical thinning distant from the tumor at the time of diagnosis. Further, morphological changes (i.e., cortical thinning in the precuneus, occipital lobe, and temporal lobe) strongly correlated with long term survival. These findings confirm the widespread impact GBM has on the brain and provide a foundation for a potentially easily acquired prognostic brain imaging biomarker.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of predicting overall survival of a glioblastoma (GBM) patient, the method comprising:
obtaining at least one morphometric image from the GBM patient;
identifying at least one radiomic biomarker based on the at least one morphometric image; and
determining an overall survival value based on the at least one radiomic biomarker.

2. The method of claim 1, wherein the at least one radiomic biomarker comprises a structural change distant from a primary tumor mass.

3. The method of claim 1, wherein the at least one radiomic biomarker comprises subcortical volume.

4. The method of claim 1, wherein the at least one radiomic biomarker comprises cortical thickness.

5. The method of claim 4, wherein the at least one radiomic biomarker comprises right precuneus cortical thickness.

6. The method of claim 4, wherein the at least one radiomic biomarker comprises temporal lobe cortical thickness.

7. The method of claim 4, wherein the at least one radiomic biomarker comprises occipital lobe cortical thickness.

8. A method of monitoring a glioblastoma (GBM) patient, the method comprising:
obtaining at least one morphometric image from the GBM patient; and
identifying at least one radiomic biomarker based on the at least one morphometric image.

9. The method of claim 8, wherein the at least one radiomic biomarker comprises a structural change distant from a primary tumor mass.

10. The method of claim 8, wherein the at least one radiomic biomarker comprises subcortical volume.

11. The method of claim 8, wherein the at least one radiomic biomarker comprises cortical thickness.

12. The method of claim 11, wherein the at least one radiomic biomarker comprises right precuneus cortical thickness.

13. The method of claim 11, wherein the at least one radiomic biomarker comprises temporal lobe cortical thickness.

14. The method of claim 11, wherein the at least one radiomic biomarker comprises occipital lobe cortical thickness.

15. A method for selecting treatments for a glioblastoma (GBM) patient, the method comprising:
obtaining at least one morphometric image from the GBM patient;
identifying at least one radiomic biomarker based on the at least one morphometric image; and
selecting one or more treatments based on the at least one radiomic biomarker.

16. The method of claim 15, wherein the at least one radiomic biomarker comprises a structural change distant from a primary tumor mass.

17. The method of claim 15, wherein the at least one radiomic biomarker comprises at least one of subcortical volume and cortical thickness.

18. The method of claim 17, wherein the at least one radiomic biomarker comprises right precuneus cortical thickness.

19. The method of claim 17, wherein the at least one radiomic biomarker comprises temporal lobe cortical thickness.

20. The method of claim 17, wherein the at least one radiomic biomarker comprises occipital lobe cortical thickness.
